(19) United States
(12) Patent Application Publication
(10) Pub. No.: US 2016/0228678 A1
(43) Pub. Date: Aug. 11, 2016

(54) Highly Durable Dual Use Catheter for Analyte Sensing and Drug Delivery

(71) Applicants: Robert S. Cargill, Portland, OR (US); William Kenneth Ward, Portland, OR (US); Joseph D. Kowalski, Portland, OR (US)

(72) Inventors: Robert S. Cargill, Portland, OR (US); William Kenneth Ward, Portland, OR (US); Joseph D. Kowalski, Portland, OR (US)

(73) Assignee: Pacific Diabetes Technologies, Portland, OR (US)

(21) Appl. No.: 14/986,469

(22) Filed: Dec. 31, 2015

Related U.S. Application Data
(60) Provisional application No. 62/099,386, filed on Jan. 2, 2015.

(51) Int. Cl. A61M 25/00; A61B 5/1486; A61B 5/145; A61M 5/14; A61B 5/00

(52) U.S. Cl. CPC A61M 25/0017; A61M 5/14; A61B 5/6852; A61B 5/14532; A61B 5/14865

(57) ABSTRACT

This invention pertains to the concept of creating a strip that contains one or more amperometric biosensing electrodes and integrating this strip into the outer wall of a hollow catheter (cannula). The electrodes can be used for continuous sensing of an analyte such as glucose and the hollow lumen can be used concurrently for delivery of a drug such as insulin. There is a risk for electrode films to break apart during impact. However, if there is a metallic foil beneath (underlying) the thin film metal electrodes, durability and fatigue resistance are markedly improved. The term “foil” indicates a metal layer that is 2-15 μm in thickness. Foils can be created by rolling, hammering, electroplating, printing, or vacuum-deposition. A foil-polymer laminate is suitable as a substrate because it permits low-cost patterning and assembly into a durable, fatigue-resistant sensor.

### CROSS-REFERENCE TO RELATED APPLICATION

This application claims the benefit of U.S. Provisional Application No. 62/099,386, filed Jan. 2, 2015.

### BACKGROUND OF THE INVENTION

There are numerous applications for analyte sensing within the field of health care. One useful configuration that has been discussed for some time is the combination of an analyte sensor with a hollow catheter for drug delivery. This configuration is particularly attractive to people with insulin-treated diabetes because such a device could reduce their percutaneous device burden. Rather than using a separate insulin infusion catheter and a continuous glucose monitor sensor, they could use a single combined device instead.

There are many different strategies for glucose sensing that could be considered for such a combined sensing catheter. Prior art exists for the use of optical sensing technologies for glucose. U.S.20130040404A1 to Crane et al teaches an optical glucose sensor built upon an optical waveguide. U.S. 20050118726 A1 to Schultz et al teaches an optical sensing method based upon a glucose-binding fusion protein. WO 2013036492 A1 to Aasmul et al teaches an optical fiber-based sensor having a hollow fiber filled with a glucose binding assay. WO 2000064492 A1 by Ballerstadt et al teaches a porous hollow sensor containing porous beads for the optical determination of analyte concentration. Alternative sensing strategies such as viscometry have also been disclosed (eg U.S. Pat. No. 6,210,326 B1 to Ehwald). However, none of these has found commercial adoption, nor are they well-suited to pairing with drug infusion in a single device.

A more common analyte sensor design is based upon the principle of amperometry, in which analytes are detected by the electrochemical conversion of the analyte of interest on the sensor surface. The sensing electrodes are commonly fabricated through the use of sputtered or evaporated thin films deposited on the surface of a substrate. Often, indicating electrodes (also known as working electrodes) are made of platinum, gold or carbon. When a positively biased indicating electrode is coupled with a reference electrode, such as silver/silver chloride, analytes can be amperometrically detected. With the addition of an enzyme layer such as glucose oxidase, a thin film sensor can be made quite specific for certain analytes. When thin films of metal electrodes are deposited on an appropriate polymer film such as polyimide, the resulting sensor has the added advantage of flexibility. Users might find a rigid catheter or needle uncomfortable or painful.

One problem with electrodes made from metallic thin films is fragility; the layers can delaminate when exposed to physical trauma such as impact, flexion, shear stresses, and tensile stresses. For example, Azoubi et al found that durability of thin film electrodes is limited. More specifically, a large number of flexion cycles led to materials failure, a phenomenon known as cycle fatigue (1). While the durability of a thin film may be sufficient for short-term applications, longer term ambulatory sensing applications require a much greater ability to withstand trauma. In the case of indwelling subcutaneous sensors, the sensor must withstand repeated flexion over a period of time lasting from 3 to 7 days or beyond. Over this extended duration, the sensor may experience thousands of bending cycles due to the movement of the patient. In the case of a long-distance runner, a sensor could easily experience 20,000-40,000 cycles over the course of a single workout alone. In Azoubi's bench top studies, thin films were shown to suffer cracking in as few as 500 cycles (1); this phenomenon is aggravated by immersion in warm, wet, high-salt environments such as those presented by mammalian blood or subcutaneous interstitial fluid. Consequently, the electrodes in the leading commercially-available CGM sensor (made by Dexcom, Inc) are constructed from durable solid wires rather than thin films. Examples of this design can be found in many patent disclosures. U.S. Pat. No. 8,812,072 B2 to Brister et al teaches a wire-based variable stiffness transcutaneous medical device. U.S. Pat. No. 8,543,184 B2 to Boock et al teaches a wire-based transcutaneous implantable continuous analyte sensor with a silicone-based membrane. U.S. Pat. No. 8,060,174 B2 to Simpson et al teaches a biointerface for a wire-based sensing electrode. U.S. Pat. No. 8,515,519 B2 to Brister et al teaches a transcutaneous analyte sensor assembly. U.S. Pat. No. 5,165,407 to Wilson et al teaches a flexible, solid wire-based glucose sensor. U.S. Pat. No. 7,471,972 B2 to Rhodes et al teaches a multi-electrode wire-based sensor. U.S. Pat. No. 9,131,885 B2 to Simpson et al teaches a multi-layer sensor having a solid core. However, a wire or rod has a solid core and is thus not compatible with drug delivery, which requires a hollow lumen. None of these devices would be suitable for combined analyte sensing and drug delivery due to their lack of a hollow lumen.

Earlier inventors have disclosed sensors coupled with hollow catheters. In U.S. Pat. No. 8,886,273 to Li, Kamath, and Yang, the inventors teach a glucose sensor disposed within a hollow catheter. More specifically, the sensor in this invention is disposed inside a larger diameter catheter that is indwelled inside a blood vessel. Whereas such an invention is appropriate for measuring a liquid (blood) that exists within a catheter, such a design is not appropriate for a sensing catheter which is intended for measuring glucose in subcutaneous fatty tissue. For use in subcutaneous tissue, the sensing elements must be on the outer wall of the hollow catheter. Stated differently, a “wire sensor within a tube” or “tube within a tube” design will not allow proper function in subcutaneous tissue. For drug delivery, the inner lumen must be hollow. Similarly, in U.S. Pat. No. 6,695,958 B1 to Adam et al, the authors disclose a device having sensing elements located in the interior of the hollow part and designed to measure analytes in the interior lumen. However, for a subcutaneous sensing catheter similar to CGM devices in common use, it is necessary to have an open interior (lumen) to allow for drug delivery into the body. In our invention, the outer wall, which is not in contact with a drug and which is bathed with glucose-containing subcutaneous interstitial fluid, is the optimal location for the sensing elements.

Other sensor configurations have been disclosed that require the withdrawal of fluid samples from the body in order for sensing to occur. U.S. Pat. No. 5,174,291 A to Schoonen et al discloses a hollow fiber-based glucose sensor that involves dialysis with a test solution. CA 2347378 A1 to Knoll et al incorporates a hollow probe for the withdrawal of interstitial fluid. EP 1327881 A1 to Beck at al teaches a hollow electrochemical cell with internal sensing elements requiring the drawing up of the fluid sample. U.S. Pat. No. 8,277,636 B2 to Sode et al teaches a glucose dehydrogenase-based sensor incorporating an interstitial fluid sampling device. U.S. 20060000710 A1 to Weidenhaupt et al teaches a method for determining glucose concentration that requires the use of a device that has an external sensor coupled with a fluid-sampling pump. U.S. 20110180405 A1 to Chinnayelka teaches a sensor incorporating a hollow member and a lancet for the sampling of interstitial fluid. U.S. Pat. No. 5,176,632 A to Bernardi teaches a system that incorporates a microdialysis-based sensor. U.S. Pat. No. 6,605,048 B1 to Levin et al teaches a sampling device that incorporates a vacuum for the drawing up of a blood sample from the skin surface. None of these would permit ongoing delivery of a drug with simultaneous exposure of the sensor to interstitial fluid. Consequently, these systems are not compatible with continuous subcutaneous drug infusion.

Other sensor configurations have been disclosed that utilize microneedles to reduce the invasiveness of the measurement technique, such as the invention that is the subject of WO2006116605 A2 to Liepmann et al. However, the chief problem with microneedle arrays is the difficulty of keeping all the microneedles indwelled in mammalian tissue during body movement. Because microneedles are short in length, some of the needles will have a tendency to come out of tissue when the person moves suddenly or forcefully. This problem of unintentional explantation renders them unsuitable for multiday use in an outpatient setting.

In order to fabricate a combined sensor/catheter, one can incorporate biosensing elements into the wall of a hollow needle or catheter. An inexpensive strategy is the fabrication of arrays of planar sensing strips which are then individualized. One sensing strip is attached to the surface of each catheter. The most obvious and simplest strategy would be to directly deposit metal (e.g. platinum, gold) indicating thin film indicating electrodes and silver reference thin film reference electrodes on the underlying polymer layer such as polyimide or polyester. Common methods of depositing the platinum and silver electrodes include sputtering, thermal evaporation, printing, silk screening, or use of an adhesive layer on a thin metal film. After deposition, the silver would subsequently be chloridized using an electrolytic procedure (electrochloridization) or by immersion in ferric chloride. This planar sensor substrate can then be wrapped around and glued to the surface of a needle or tube in order to integrate the sensor with the drug delivery catheter. The use of a flexible metalized substrate has substantial advantages in terms of low cost of production, as hundreds of devices can be manufactured in batch processing using photolithographic techniques, mature technologies developed over several decades by the electronics industry. One such design, disclosed in WO2002039086 to Ramey et al, incorporates printed electrode films. However, after carrying out many studies in animals, we have observed a major problem with sensing catheters made of thin film metal electrodes deposited over a polymeric layer. These sensors exhibited frequent delamination and general lack of durability. This invention teaches methods by which the durability of sensing catheters can be markedly improved.

### SUMMARY OF THE INVENTION

This invention pertains to the concept of creating a sheet or strip that contains one or more amperometric biosensing electrodes and integrating this sheet or strip into the outer wall of a hollow catheter (cannula). When thin film metal electrode materials are placed directly over polymeric surfaces (with or without underlying thin adhesion layers) the device becomes fragile. The electrode films and other elements of the sensor often delaminate or break apart during impact, and therefore, such a device is not adequate for use as an indwelling catheter. In fact, substantial electrode delamination can be seen after only a few hours of in vivo use. In the experience of the inventors, whether or not a 100 nm tie (adhesion) layer of gold is deposited under the electrodes, such a design leads to a frequent separation of the gold layer from the polyimide, frequent separation of the platinum or silver electrode films from the gold layer, and frequent fragmentation of the metal layers.

Thus, an improved sensor composition is required in order to enhance durability. During exploration of alternative designs, we found that inclusion of a metallic foil beneath (underlying) the thin film metal electrodes markedly improved durability and fatigue resistance, while maintaining sufficient flexibility for fabrication and use as a biosensor in mammals. The use of the term “foil” indicates a metal layer that is at least 2 micrometers (μm) in thickness, that is, much thicker than the thin film layer typically deposited by sputtering, evaporation, printing or electroplating. Foils are usually made by rolling a metal stock through a pair of hardened metal rolls. Hammering of the stock is an alternative way of making the foil. Alternatively, metal foils can be made by electroplating, sputtering, thermal evaporation and deposition of metal inks. Rolled or hammered metal foils have very high internal cohesive forces. More specifically, the process of rolling achieves a tightly-packed crystal and grain structure which increases strength. If necessary, the rolled metal can be annealed to reduce brittleness and reform the natural grain structure. Discussions of the beneficial mechanical properties of foils can be found in three scientific articles listed elsewhere in this document and attached (1-3).

For these reasons, a metal foil (underneath the thin electrode film) is well-suited for the purpose of durability as described in this invention. It is well known that for the materials of a biosensor to be sufficiently durable, all layers must have a high degree of adhesion to the adjacent layers.

In our studies, we found that the presence of an underlying metal foil dramatically enhanced resistance to cyclic fatigue. More specifically, we found that the physical integrity of a foil having a thickness of 2-15 μm was orders of magnitude greater than a sputtered film (unattached to an underlying foil) with a thickness of 50-100 nm. Of course, in order to yield a functioning sensor, there is a need for the foil and associated layers to undergo a process of patterning (to create the dimensions for the indicating and reference electrodes, the contact pads and the interconnect traces). A foil-polymer laminate was chosen as a substrate that would permit low-cost patterning and assembly into a durable, fatigue-resistant sensor.

### BRIEF DESCRIPTION OF DRAWINGS

FIG. 1, a cross-section, shows layers of a sensing catheter whose design makes it susceptible to cyclic fatigue. The outer layer 1 is composed of sensing membranes that include an enzyme and other materials such as a redox mediator and a permselective polymer layer. In deeper layers, there is a layer of thin film metal electrode 2. Depending on the location of the cross-section, the electrode 2 can be an indicating, reference electrodes, or counter-auxiliary electrode. Deeper still is a thin film adhesion layer 3 (such as gold); a polymer layer 4 (such as polyimide); an adhesive layer 5; a tube 6 such as a stainless steel tube; and a central opening or lumen 7. When sensing catheters fabricated with this design are subcutaneously placed in mammals, there are frequent occurrences of delamination of certain junctions or fragmentation of certain layers, designated here with the label “fragile interface.”

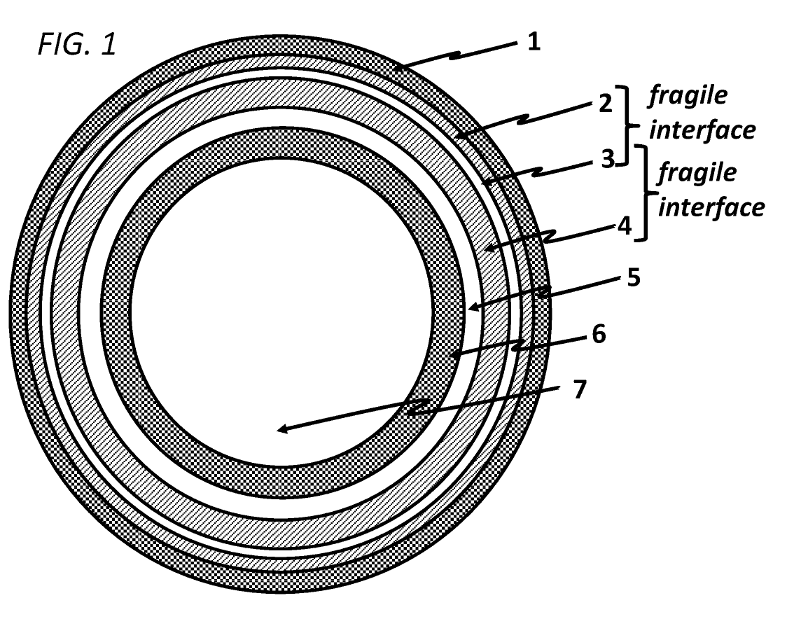

FIG. 2, a cross-section, shows another example of a sensing catheter whose design makes it susceptible to cyclic fatigue. In this figure, no adhesion layer is present and the thin film metal electrode layer 2 is directly deposited on to a polymer substrate 4 such as polyimide. The label “fragile interface” denotes the specific junction that is not durable.

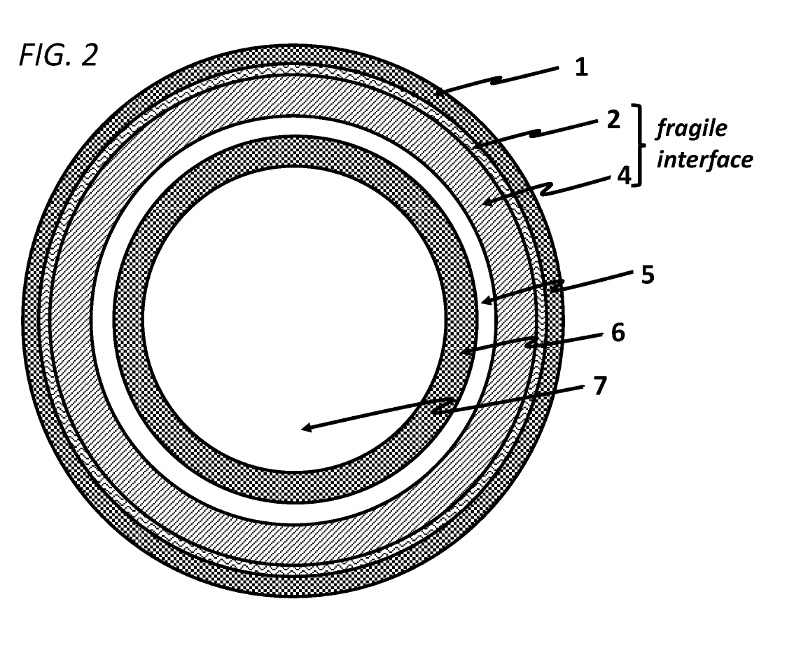

FIG. 3, a cross-section, shows the layers of a rigid sensing catheter whose design is optimized for durability. Of note is the presence of a metal foil 8, such as titanium foil, that underlies the thin film metal electrode 2. An adhesive 9, such as B-stage acrylate adhesive, adheres the foil 2 to the underlying polymer layer 4. A high tack-strength adhesive 10 adheres the polymer 4 to the stainless steel tube 6.

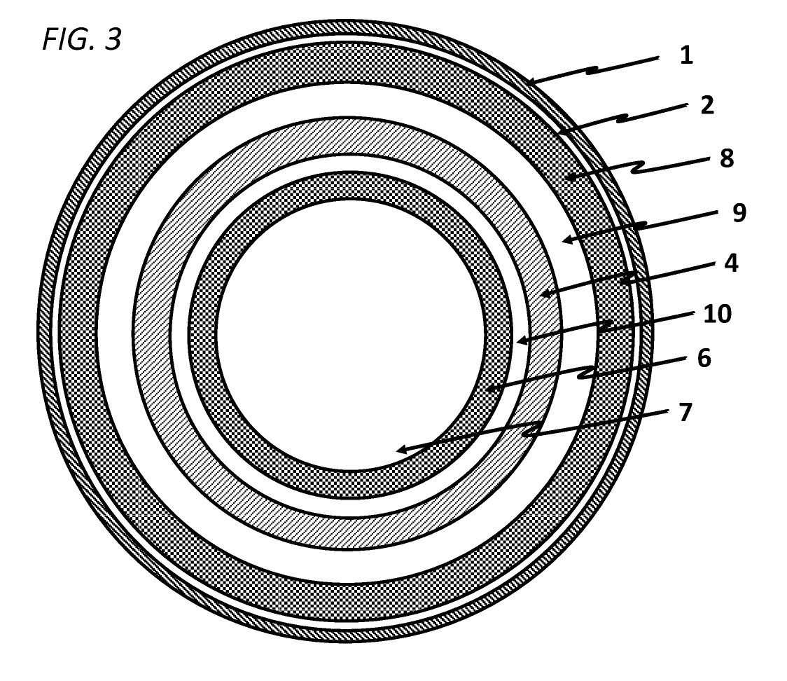

FIG. 4, a cross-section, shows the layers of a flexible sensing catheter whose design is optimized for durability. The layers are similar to those of FIG. 3, except that the substrate tube 11 is made of a flexible polymer rather than a rigid material.

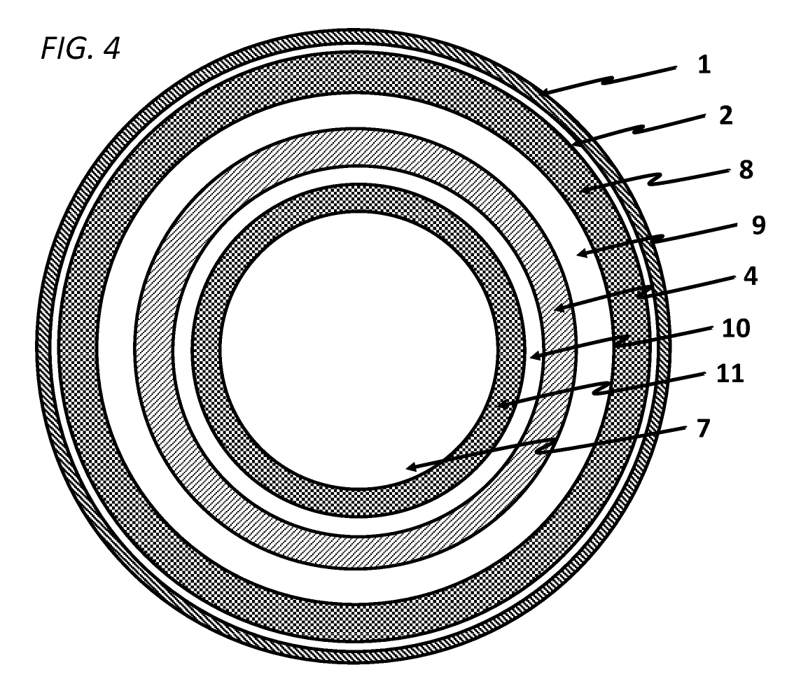

FIG. 5 (bottom panel) shows an array 12 that consists of sixteen tri-electrodes prior to their separation and individualization. FIG. 5 (top panel) shows the distal part of one tri-electrode after it has been individualized and adhered to the outer wall of a rigid tube 13. In order to provide increased sensing accuracy by use of redundant signal collection, there are three indicating electrodes (distal electrode 14, middle electrode 15 and proximal electrode 16). The reference electrode 17 interdigitates with the indicating electrodes. The interconnect traces of the indicating and reference electrodes 18 travel proximally and terminate in contact pads (not shown), which serve to electrically connect all electrodes with a body worn electronic unit.

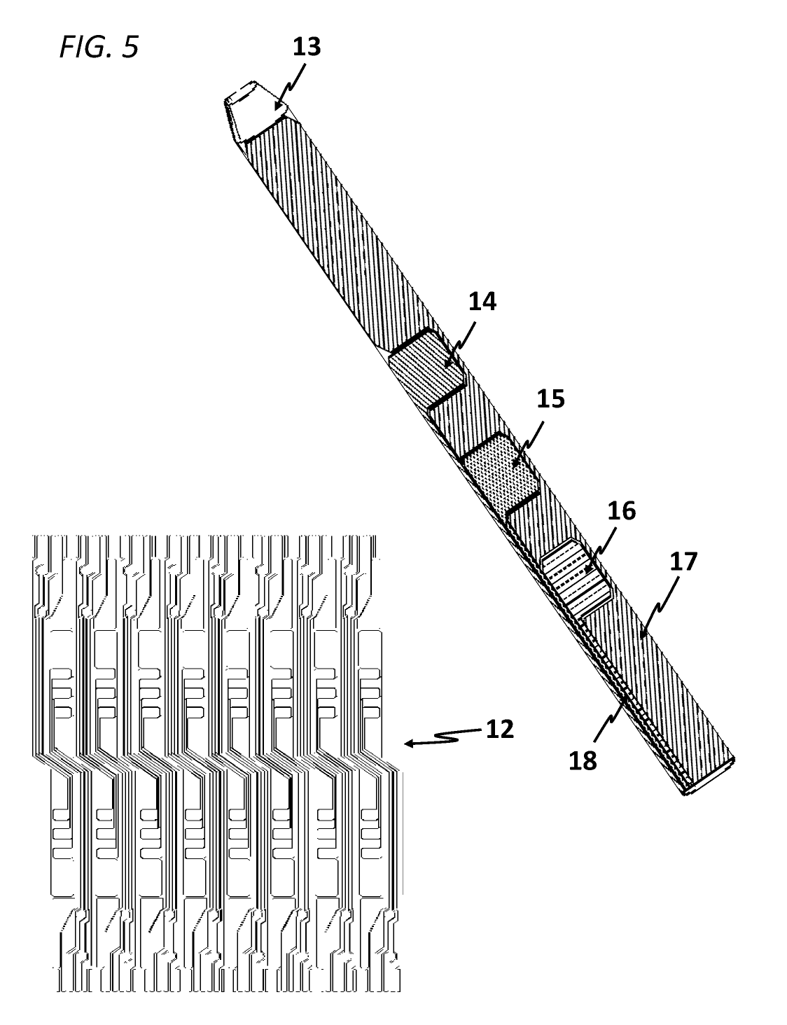

### DETAILED DESCRIPTION

In addition to durability, the cost of construction is important. The mass of the expensive indicating electrode metal (e.g. platinum, gold or carbon) must be minimized in order to yield a commercially viable solution. Thus, a thin film is favored for the choice of the indicating electrode. Because the underlying metal foil is thicker (greater mass), it must be a low cost material. Although one could directly laminate a thick platinum foil to the polymer, the cost of such a platinum-rich device would be prohibitive for a commercially viable disposable medical device. We discovered that the use of inexpensive titanium foil as an interfacial layer between the polymer and the electrode thin film serves as a low cost solution to the problem of fragility that was observed without use of a foil. In sensing catheters removed from active pigs, we observed very good mechanical integrity when titanium foil was utilized. More specifically, there was no metal fragmentation and no separation at the following interfaces: (1) the junction of the platinum or silver electrodes and the underlying titanium foil, (2) the junction of the titanium foil and the underlying B-stage acrylate adhesive, and (3) the junction of the acrylate adhesive and the underlying polyimide substrate.

All layers of the sensing catheter must be tightly adhered to the adjoining layers. One method of creating interfaces with good adhesion and good durability is the use a laminating press. To laminate the foil (e.g. titanium) to the underlying polyimide polymer, one can use a hydraulic press set to a high temperature (for example, 375 deg F.) and high pressure (for example, 235 PSI). A high tack adhesive such as B-stage acrylate is located at the interface of the foil and polymer and adheres the two materials together. After the lamination, thin film electrode materials can be deposited over the durable metal foil. The thickness of the metal foil is typically 2-15 μm.

The specific nature of how different materials interact with one another is pertinent to this invention. For example, despite the fact that the internal cohesion of the platinum and silver thin film is poor, both films adhere tightly to the underlying foil layer because of the high degree of similarity in the mechanical properties of the two metal layers. This adhesion was found to be far superior to that of adhesion to any polymer layer tested. Furthermore, the cohesion within the metal layer of the foil itself prevents it from breaking down into small fragments, a fate to which thin films quickly succumb under stress. This offers the benefit of cooperativity, i.e., removal of any small element of foil from the surface of the polymer requires the breaking of the bond formed by the entire surface area of the foil. This property stands in contrast to the removal of fragments of a thin film, which require much lower forces due to the smaller surface area of the fragments. The adhesion of the metallic foil to the polymer below is also improved dramatically in comparison to a thin-film/polymer adhesion.

The metal of which the foil is composed must be chosen carefully. In the case of an amperometric glucose sensor, the indicating electrode is typically platinum, gold or carbon. Copper (which is commonly used as the foil for flexible electronic circuits), is not suitable for use in a biosensor. Specifically, if there is concurrent physical contact between interstitial fluid, copper and platinum, a large galvanic current will occur as a result of a dissimilar metal junction. An ideal candidate for the foil is titanium, which is inexpensive and which we found to cause little to no galvanic current when paired with platinum. Silver and copper are not suitable as this foil material. Gold is of intermediate value.

### EXAMPLE 1

### Step 1

### Laminate Metal Foil to Polymer Substrate

### Purpose

This step creates a laminate of titanium and polyimide (Ti/Pi). In this example, the Ti thickness is 5 μm and the polyimide thickness is 12.5 μm, though these dimensions should not be construed as limiting. This example creates a laminate rectangle whose dimensions are 60 mm×85 mm.

### Equipment

Heated hydraulic press capable of achieving 400 deg F.;

### Materials

DI water; Polyimide sheet w/b-stage acrylate adhesive; Titanium foil; press pads; and graphite press plates.

### Plate Setup Process

Between the caul plates of the hydraulic press, materials should be stacked in the following order, from bottom to top: Graphite press plate; press pad; Titanium foil; Polyimide, with b-stage adhesive facing titanium foil; press pad; Graphite press plate.

Prepare graphite plate, graphite foil, and Teflon sheets prior to handling polyimide and titanium. All sheets should be cut to the size of the caul plates and cleaned with IPA, followed by careful inspection for lint or contaminants. If any portion cannot be cleaned properly it should be discarded and replaced.

Set titanium on a Teflon sheet atop graphite plate/foil. Inspect for lint or contaminants. Never apply any chemical to the b-stage adhesive, it should only be cleaned using bottled gas, clean compressed air, or a non-linting wipe.

Set polyimide sheet, with its plastic release layer (if present) removed, on top of titanium foil, b-stage adhesive facing downward. Look through the polyimide for any particles which may be lodged between sheets. If any appear, remove the polyimide and clean both sheets.

### Press Operation

### Place plate stack into hydraulic press and apply 5000 lb of force to caul plates. Set temperature setting to 375 deg F. for both top and bottom plates.

Once both caul plates reach 375 deg F., set press to 15000 lb and leave in place for 1 hour. Turn off heaters and allow caul plates to cool to under 100 F, then remove plate stack from press. Regions that are visibly wrinkled or that have contaminants are not suitable for sensor production.

### General Equipment and Supplies (for All Following Steps)

Double-sided polyimide tape; plastic card; razor blade; 50×75 mm glass slide; isopropyl alcohol (IPA); deionized (DI) water; Pt (platinum) target; Ag (silver) target; aluminum foil; Ar plasma etcher; quartz crystal microbalance (QCM); sputter tool; hot plate; mask aligner—e.g OAI 200 tabletop mask aligner; spin coater capable of 300 RPM; argon source.

### Step 3

### Prepare Ti/Pi Laminate for Application of Pt and Ag Electrodes

Clean glass slide using soap and tap water, IPA wash, DI rinse, Ar plasma clean for 1 minute. Blow dry with clean air, argon, or nitrogen gas. Place sheet of aluminum foil on cutting board for use as workspace. Cut a 60 mm×85 mm rectangle of polyimide tape to allow for misalignment. Slowly apply double-sided polyimide tape onto the glass, ironing bubbles out using the plastic card as it is applied. Cut excess tape from slide, being sure to leave no exposed glass around the edges to accommodate the entirety of the pattern. Cut out a slightly oversized piece of Ti/PI laminate and iron on the laminate to the slide using plastic card. Discard if laminate is creased.

### Step 4

### Deposition of Silver Film

### Purpose

To deposit a layer of Ag (later chloridized to Ag/AgCl) in order to create reference electrode. Nominal thickness is 400 nm, to allow for a reasonable thickness of Ag/AgCl after chloridization (chloridization reduces the thickness of Ag). In this process, silver sputtering is used, but other methods such as thermal evaporation, printing, or electroplating can also be used.

### Specific Materials

### Treated 50×75 mm Ti/PI sheet on glass slide, CRC-100 sputter unit, Ag target.

### Method

Cut two small tabs of double-sided tape and place them on the bottom of the substrate to prevent it from sliding due to pump vibration or gusts of air when the roughing pump is turned on. Place substrate in CRC-100 unit, turn on pumps. Leave system to pump down for 15 minutes. This degasses any exposed polyimide/adhesive and improves vacuum quality. Sputter until Quartz Crystal Microbalance (QCM) reading is 5.00 kA (500 nm) of Ag. (Gain=75, Density=10.5, Z-ratio=0.529, Tooling Factor=256). Remove from CRC-100 unit, being exceedingly careful to not contact the silver coating. Silver thin films are extremely prone to scratching and should never be scrubbed. If cleaning must occur, proceed with a first-surface optics cleaning process. Tape test in a corner with 3M Magic Scotch tape to ensure good adhesion. Store in a dust-free covered container.

### Step 5

### Ag Patterning and Etch (Remove Unwanted Ag)

Purpose—To pattern photoresist for Ag pads on Ti/PI substrate.

### Specific Materials

50×75 mm Silver sputtered Ti/PI substrate on glass slide; NaOH pellets; 300 mL beaker; 250 mL beaker; optical mask, S1813 (photoresist); 80/20 HDMS primer.

### Materials and Equipment (for Cleanroom Use)

Mask aligner; Spinner; hotplate; DI water; scale; S1800 series photoresist; NaOH (pellets or solution).

### Method

Carry out photoresist process that is included at the end of this document.

Mix Ag etch solution. Add 75 mL of 3% USP grade H202, then 8 mL laboratory grade 30% Ammonium Hydroxide to a crystallizing dish. Immerse patterned substrate in solution for 30 seconds, gently agitating. Bubbles will not form when the reaction is complete. It is important to note that it is exceedingly critical that this etch completes. Rinse with DI water and blow dry with nitrogen gas or Argon. Remove photoresist with 0.3M NaOH solution.

### Step 6

### Pt Patterning, Sputtering, and Liftoff

### Purpose

To pattern Pt pads on Ti/PI/Ag substrate.

### Specific Materials

50×75 mm Silver sputtered Ti/PI substrate on glass slide; NaOH pellets; 300 mL beaker; 250 mL beaker; optical mask; S1813 primer; Ti/PI/glass with Ag deposited on surface; 80/20 primer; Ag etch film mask; Borax; 3 mL pipette; Acetone; isopropyl alcohol (IPA); crystallizing dishes; graduated cylinder; timer.

### Method

Carry out photoresist process that is included at the end of this document. Mark the mask name and revision on the traveler document. Protect using a cleanroom wipe or Kimwipe. Keeping the substrate dust-free is critical. Clean under Ar for 1 minute. Place into vacuum system, turn on pumps, allow to pump for 15 minutes. Sputter 90 nm (0.900 KA) Pt. (Gain=75, Density=10.5, Z-ratio=0.529, Tooling Factor=256). Use 3 strips of Scotch tape to cover the entirety of the substrate. Press down firmly across the entirety of the substrate, then slowly remove in order to remove platinum layer. Inspect tape-test sheet for any failures in Pt adhesion. Mark any failures in traveler, label and keep the test if failures are found. Use an additional piece of tape to remove any bridges between platinum pads. (These will have a different appearance than the pad themselves and are quite noticeable). Remove photoresist/remaining Pt by tape method (3m magic tape over entire array), then sonication in 0.5M NaOH. If any bridges remain, gently scrub using Kimwipe while in solution.

### Step 7

### Titanium Etch (Remove Unwanted Ti in Order to Create Electrical Interconnects)

### Purpose

To define titanium traces on sensor.

### Specific Materials

Ti/Pi mounted slide; titanium etchant; 400 mL beaker; crystallization dish; DI water; NaOH.

### Equipment

Ultrasonic cleaner

### Method

Carry out photoresist process that is included at the end of this document. Prepare etchant bath. Place substrate in etchant solution and observe closely, rinse with DI water when etch is complete.

Rinse with DI water and blow dry with nitrogen gas or argon.

### Step 8

### Prepare Sensors For Human Use

### Individualize, Wrap, Chloridize, Apply Protective Coat to Reference Electrode, and Clean Indicating Electrodes

Apply 5 mil (0.005 inch) polyimide backer strip with adhesive to back side of the electrode array (back side is the side without electrodes). Then apply protective tape to photoresist-covered front side (for example, S2020 tape from Champion). Individualize the 3-electrode strip by use of an arbor press.

Wrap the strip around a 21-25 gauge stainless steel needle (sharp bevel on end) or blunt tube. Sensing strips are wrapped axially around the needle/tube and adhered using epoxy or other biocompatible adhesive. If a blunt tube can be used, a sharpened stylet within the tube is utilized in order to pierce the skin upon insertion. (The stylet is later removed, allowing drug delivery via the lumen of the tube).

Ferric chloridize with 50 mM FeCl₃ for 3 min. ALTERNATIVE: Electrochloridize at 0.6 V×10 min using power supply configures so that the Ag is the Anode (+) and Pt is the cathode (−). Bath for electrochloridization is KCl and HCl, both 0.5 M.

Coat reference electrode with 5% polyurethane in 95%-5% THF-DMAC; dry×20 min at 40 deg C.

Voltage cycle (clean) indicating electrodes in 1×PBS, −1.5 volts×5 min, 1.5 volts×5 min, −1.5 volts×5 min.

### Apply Enzyme Layer and Outer Membrane

Drop cast with glucose oxidase (GOX), bovine serum albumin (or human serum albumin) and glutaraldehyde in weight ratio of 6:4:5 or 6:4:1; then dry for 10 or more min at 40° C. NOTE: The purpose of the glutaraldehyde is to crosslink and immobilize the enzyme/albumin. Deposit additional GOX layers as desired, for example, four more times (5 total coats). Dry all but final coat for 10 min and final coat for 20 min. Then rinse in stirred DIW for 10-15 minutes. Use Kimwipe to remove GOX flaps/strings that are not well-adhered. Coat twice with 1.5-2.5% w/v polyurethane (PU) on the IE (indicating electrodes). Alternatively use a PU that includes silicone and/or polyethylene oxide moieties in order to regulate oxygen and glucose permeation, respectively. Solvent: 95-5 THF-DMAC. Dry each PU coat×20 min at 40 deg C. Keep solvent and polymer/solvent dry with molecular sieves 3A or 4A.

### Assemble

Insert the sensing catheter into a battery powered telemetry module (low energy Bluetooth module such as that marketed by Nordic, Inc).

### Sterilize

Expose to e-beam, gamma irradiation, ethylene oxide or activated glutaraldehyde sterilizing solution.

### Attach to Insulin Pump and Operate Device

After priming with insulin, an infusion line from an insulin pump (e.g. Medtronic Minimed, Animas Ping, Tandem t-slim, Roche Spirit, etc) is attached to the sensing catheter (which is located in subcutaneous tissue) and insulin is delivered. The constant pressure head from the fluid infusion line prevents fluids from coming back out of the body. In order to be displayed to the user, the glucose concentration or the electrical current or voltage data representing glucose concentration is obtained from the sensor. These data are transmitted by Bluetooth or other wireless protocol to the display of the insulin pump, to a computer, to a dedicated medical device, or to a cell phone. Storage of data can be carried out on any of these devices or on the body worn electronics unit that directly interfaces with the subcutaneous sensing catheter. An advantage of storing the glucose data on the body-worn unit is that the data are not lost if the receiving unit is lost or out of range.

### Appendix

### Photoresist Process (Common to Multiple Steps)

### Materials

50×75 mm Ti/PI substrate on glass slide; NaOH pellets or solution; 300 mL beaker; 250 mL beaker; optical mask; photoresist.

### Method

Mix 200 ml 0.15M NaOH (8 g/L w/pellets or 15 mL/L w/10M solution) primary developer in glass dish. Ensure that solution is well mixed, especially if using NaOH pellets. Use bath for no more than 2 developments. Mix 0.075M NaOH secondary rinse in glass dish. Ensure that solution is well mixed. Spin coat two layers of photoresist. Develop in 0.15M NaOH developer, gently agitating. Rinse in secondary bath for 10 seconds. Dry with nitrogen gas, inspect for developed regions with remaining resist. (Exposed regions should appear uniform across the entirety of the substrate. Properly cleaned regions will gain a faintly white appearance as they go from wet to try if no photoresist remains on the surface). If regions remain, immerse in primary and secondary baths for an additional 5 seconds and check again. If substantial regions remain, air dry, clean with 0.3M NaOH, and return to step 4. Check process parameters. Bake for 60 seconds as above and allow to cool.

### CITED REFERENCES (ATTACHED)

1. Alzoubi K, Lu S, Poliks M. Experimental and Analytical Studies on the High Cycle Fatigue of Thin Film Metal on PET Substrate for Flexible Electronics Applications. IEEE Transactions on Components, Packaging, and Manufacturing Technology. 2011; Vol 2.

2. Dai C, Zhang R, Yan C. Size effects on tensile and fatigue behaviour of polycrystalline metal foils at the micrometer scale. Philosophical Magazine. 2011; 91:932-45.

3. Lavvafi H, Lewandowski J R, Lewandowski J J. Flex bending fatigue testing of wires, foils and ribbons. Materials Sci and Engineering 2014; 1:123-30.

We claim:

1. A single-walled hollow catheter, whose indwelled length is 5-15 mm, that allows simultaneous analyte sensing within, and passage of a liquid drug into, a mammalian body,
wherein the outer wall of said catheter includes one or more indicating electrode films (film thickness <500 nm) and one or more reference electrode films (film thickness <900 nm),
wherein an enzyme layer is located external to the outer surface of said indicating electrode,
wherein said indicating and reference electrodes are in direct contact with an underlying metal foil, said foil being internal to said electrodes, and
wherein said foil is in contact with an underlying polymer layer, said polymer layer being internal to the foil.

2. The device of claim 1 in which the catheter is flexible.

3. The device of claim 1 in which the catheter is rigid.

4. The device of claim 1 in which the indicating electrode films are composed of one or more of the following: carbon, platinum, gold, other platinum group metal, or metal oxide.

5. The device of claim 1 in which the reference electrode film includes silver and/or silver chloride.

6. The device of claim 1 in which the analyte is glucose.

7. The device of claim 1 in which the drug is insulin.

8. The device of claim 1 in which the enzyme is glucose oxidase or glucose dehydrogenase.

9. The device of claim 1 in which a polymeric permselective membrane is disposed external to the enzyme.

10. The device in claim 1 in which said device is coupled with an electronic module which provides a bias voltage to, and measures analyte responsive currents from, the sensing catheter.

11. A method for the simultaneous measurement of a subcutaneous analyte concentration and continuous drug infusion through the use of a single-walled hollow catheter, whose indwelled length is 5-15 mm, and a drug pump,
wherein retrograde flow (escape from the body) of interstitial fluid or blood is prevented by its connection with said pump that maintains a constant positive pressure,
wherein the outer wall of said catheter includes one or more indicating electrode films (film thickness <500 nm) and one or more reference electrode films (film thickness <900 nm),
wherein an enzyme layer is located external to the outer surface of said indicating electrode,
wherein said indicating and reference electrodes are in direct contact with an underlying metal foil, said foil being internal to said electrodes, and
wherein said foil is in contact with an underlying polymer layer, said polymer layer being internal to the foil.

12. The device of claim 11 in which the catheter is flexible.

13. The device of claim 11 in which the catheter is rigid.

14. The device of claim 11 in which the indicating electrode films are composed of one or more of the following: carbon, platinum, gold, other platinum group metal, or metal oxide.

15. The device of claim 11 in which the reference electrode film includes silver and/or silver chloride.

16. The device of claim 11 in which the analyte is glucose.

17. The device of claim 11 in which the drug is insulin.

18. The device of claim 11 in which the enzyme is glucose oxidase or glucose dehydrogenase.

19. The device of claim 11 in which a polymeric permselective membrane is disposed external to the enzyme.

20. The device in claim 11 in which said device is coupled with an electronic module which provides a bias voltage to, and measures analyte responsive currents from, the sensing catheter.
